(19) United States
(12) Patent Application Publication
(10) Pub. No.: US 2023/0270421 A1
(43) Pub. Date: Aug. 31, 2023

(54) SPECIMEN RETRIEVAL DEVICE

(71) Applicant: Covidien LP, Mansfield, MA (US)

(72) Inventors: Garrett P. Ebersole, Hamden, CT (US); Jacob C. Baril, Norwalk, CT (US); Nicolette LaPierre Roy, Windsor Locks, CT (US); Justin J. Thomas, New Haven, CT (US); Matthew A. Dinino, Newington, CT (US); Saumya Banerjee, Southington, CT (US); Roy J. Pilletere, Middletown, CT (US)

(21) Appl. No.: 18/172,575

(22) Filed: Feb. 22, 2023

Related U.S. Application Data
(60) Provisional application No. 63/314,636, filed on Feb. 28, 2022.

(51) Int. Cl. A61B 17/00

(52) U.S. Cl. CPC A61B 17/00234; A61B 2017/00287; A61B 2017/00867

(57) ABSTRACT

A specimen retrieval device for endoscopic procedures includes a sleeve, barbed sutures, guide members, and a contractible band that is supported about an open distal end of the sleeve. Each of the barbed sutures have an elongate body, a distal portion, a proximal portion, and barbs that extend proximally from the elongate body. The guide members are secured to an inner surface of the sleeve and slidably receive respective barbed sutures. A tissue specimen to be removed from a patient is placed within a cavity defined by the sleeve and within the confines of the barbed sutures. Proximal portions of the barbed threads can be independently or simultaneously pulled in a proximal direction to withdraw the tissue specimen and the sleeve from the body cavity.

### CROSS-REFERENCE TO RELATED APPLICATION

This application claims the benefit of and priority to U.S. Provisional Patent Application No. 63/314,636, filed Feb. 28, 2023, the entire contents of which is incorporated by reference herein.

### FIELD

This disclosure relates to a surgical device for use in minimally invasive surgical procedures, such as endoscopic and/or laparoscopic procedures and, more particularly, to a specimen retrieval device for removal of tissue from a patient.

### BACKGROUND

Minimally invasive surgery, such as endoscopic surgery, reduces the invasiveness of surgical procedures. Endoscopic surgery involves surgery through small incisions in body walls, for example, viewing and/or operating on the ovaries, uterus, gall bladder, bowels, kidneys, appendix, etc. There are many common endoscopic surgical procedures, including arthroscopy, laparoscopy, gastroentroscopy, and laryngobronchoscopy, just to name a few. In these procedures, trocars are utilized for creating incisions through which the endoscopic surgery is performed. Trocar tubes or cannula assemblies are extended into and left in place in the abdominal wall to provide access for endoscopic surgical tools. A camera or scope is inserted through a cannula assembly to permit the visual inspection and magnification of the body cavity. The surgeon can then perform diagnostic and/or therapeutic procedures at the surgical site with the aid of specialized instrumentation, such as forceps, specimen retrieval devices, cutters, applicators, and the like, which are designed to fit through additional cannulas.

Several minimally invasive surgical procedures require bulk removal of body tissue or organs through a small surgical opening. Improved devices for bulk removal of tissue remain desirable.

### SUMMARY

This disclosure is directed to a specimen retrieval device for removing large tissue specimen from a body cavity during endoscopic surgical procedures. The specimen retrieval device includes a sleeve having open distal and proximal ends, barbed sutures that are slidably attached to an inner surface of the sleeve, and a band that is transitionable between open and contracted configurations to close or substantially close the open distal end of the sleeve. The barbed sutures are independently movable within the sleeve to withdraw portions of the tissue specimen through an access port during an endoscopic surgical procedure.

Aspects of the disclosure are directed to a specimen retrieval device including a sleeve, a band, and barbed sutures. The sleeve defines a cavity and has a first open end, a second open end, and an inner surface that defines the cavity. The band is supported about the first open end of the sleeve and is transitionable from an open configuration to a contracted configuration. The barbed sutures extend through the sleeve. Each of the barbed sutures has an elongate body, a distal portion, a proximal portion, and barbs that are spaced along at least a portion of the elongate body. Each of the barbed sutures is coupled to the inner surface of the sleeve and extends from the first open end of the sleeve to the second open end of the sleeve.

In aspects of the disclosure, the specimen retrieval device includes guide members that are secured to the inner surface of the sleeve.

In some aspects of the disclosure, each of the guide members defines a channel that receives one of the barbed sutures to couple the barbed suture to the inner surface of the sleeve.

In certain aspects of the disclosure, the sleeve includes a distal portion and a proximal portion, and the guide members are supported on the distal and proximal portions of the sleeve.

In aspects of the disclosure, each of the guide members on the distal portion of the sleeve is longitudinally aligned with one of the guide members on the proximal portion of the sleeve.

In some aspects of the disclosure, the guide members that are longitudinally aligned on the distal and proximal portions of the sleeve receive one of the barbed sutures such that the one of the barbed sutures extends longitudinally along the inner surface of the sleeve.

In certain aspects of the disclosure, the band is formed from a shape memory material that transitions from the open configuration to the contracted configuration in response to a change in ambient conditions.

In aspects of the disclosure, the shape memory material is selected to transition from the open configuration to the contracted configuration in response to a change temperature.

In some aspects of the disclosure, the barbed sutures include from about 4 to about 12 barbed sutures.

In certain aspects of the disclosure, the barbed sutures include from about 6 to about 10 barbed sutures.

In aspects of the disclosure, the plurality of barbed threads is formed of a biocompatible material selected from polymeric materials derived from lactones, carbonates, dioxanones, ethylene glycol, ethylene oxide, ester amides, γ-hydroxyvalerate, β-hydroxypropionate, alpha-hydroxy acid, hydroxybutyrates, poly (ortho esters), hydroxy alkanoates, polyurethanes, polyanhydrides, copolymers thereof, or combinations thereof.

In some aspects of the disclosure, the shape memory material is nitinol.

In certain aspects of the disclosure, the barbed sutures are spaced about the inner surface of the sleeve.

In aspects of the disclosure, the sleeve is formed of a biocompatible material selected from nylon, polyurethane, and latex.

In some aspects of the disclosure, the barbs of the barbed suture have a width that greater than a size of the channel defined by the guide members.

In certain aspects of the disclosure, the distal portion of each of the barbed sutures extends from the first open end of the sleeve and the proximal portion of each of the barb sutures extends from the second open end of the sleeve.

In aspects of the disclosure, each of the barbs extends from the elongate body of the barbed suture in a proximal direction.

Other aspects of the disclosure are directed to a method of removing a tissue specimen from a patient that includes introducing a specimen retrieval device having a sleeve with open distal and proximal ends and barbed sutures through an access port into a body cavity of a patient in a folded or rolled configuration; unfolding or unrolling the sleeve and barbed sutures of the specimen retrieval within the body cavity of the patient; positioning a tissue specimen into a cavity defined by the sleeve through an open distal end of the sleeve within confines of the barbed sutures; moving the open distal end of the sleeve from an open configuration to a contracted configuration; and pulling a proximal portion of at least one of the barbed sutures so that barbs of the barbed sutures engage the tissue specimen and pull at least a portion of the tissue specimen through the access port.

In aspects of the disclosure, the method includes repeating the step of pulling the proximal portion of at least one of the barbed sutures until each of the barbed sutures is pulled proximally to remove the sleeve and the tissue specimen through the access port from the body cavity.

In some aspects of the disclosure, pulling the proximal portion of at least one of the barbed sutures includes pulling the proximal portions of all the barbed sutures simultaneously.

In certain aspects of the disclosure, pulling the proximal portion of at least one of the barbed sutures includes pulling the proximal portions of two diametrically opposed barbed sutures simultaneously.

Other aspects, features, and advantages will be apparent from the description, the drawings, and the claims that follow.

### BRIEF DESCRIPTION OF THE DRAWINGS

Various aspects of the disclosed specimen retrieval devices and methods are described herein below with reference to the drawings, wherein:

FIG. 1 is a side perspective view of a specimen retrieval device according to aspects of the disclosure;

FIG. 2 is a cross-sectional view taken along section line 2-2 of FIG. 1;

FIG. 3 is an enlarged view of the indicated area of detail shown in FIG. 1 illustrating a guide member of the specimen retrieval device shown in FIG. 1;

FIG. 3A is side perspective view of an alternative version of the guide member of the specimen retrieval device shown in FIG. 1;

FIG. 4 is an enlarged view of the indicated area of detail shown in FIG. 2;

FIG. 5 is a side perspective view of the specimen retrieval device shown in FIG. 1 as the specimen retrieval device is inserted into a body cavity through a cannula positioned within an incision in a patent with barbed sutures of the specimen retrieval device positioned about a tissue specimen;

FIG. 6 is a side perspective view of the specimen retrieval device shown in FIG. 1 with the specimen retrieval device partially inserted into the body cavity through the cannula positioned within the incision in the patent with the barbed sutures and a sleeve of the specimen retrieval device positioned partially about the tissue specimen;

FIG. 7 is a side perspective view of the specimen retrieval device shown in FIG. 1 with the specimen retrieval device partially inserted into the body cavity through the cannula positioned within the incision in the patent with the barbed sutures and the sleeve of the specimen retrieval device positioned about the tissue specimen and a band at one end of the sleeve in a contracted configuration;

FIG. 8 is a cross-sectional view taken along section line 8-8 of FIG. 7;

FIG. 9 is a cross-sectional view as shown in FIG. 8 as the selective ones of the barbed sutures of the specimen retrieval device are retracted to remove a portion of the tissue specimen from the body cavity through the incision;

FIG. 10 is a side perspective partial cross-sectional view of the specimen retrieval device as the specimen retrieval device is withdrawn from the body cavity through the incision;

FIG. 10A is a side perspective view of an alternate version of the band of the specimen retrieval device shown in FIG. 1 with the band in the contracted configuration;

FIG. 10B is a side perspective view of the band shown in FIG. 10A in the open configuration;

FIG. 11A is a side perspective view of another alternate version of the band of the specimen retrieval device shown in FIG. 1 with the band in the contracted configuration; and

FIG. 11B is a side perspective view of the band shown in FIG. 11A in the open configuration.

### DETAILED DESCRIPTION

The disclosed specimen retrieval device will now be described in detail with reference to the drawings in which like reference numerals designate identical or corresponding elements in each of the several views. However, it is to be understood that the aspects of the disclosure described herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician in its customary fashion. In addition, the term “minimally invasive” is used generally to refer to endoscopic, laparoscopic, arthroscopic, and/or any other procedure conducted through a small diameter incision or cannula. Further, the term “clinician” is used generally to refer to medical personnel including doctors, nurses, and support personnel.

The disclosure provides a specimen retrieval device for use in minimally invasive surgical procedures. Aspects of the disclosure may be modified for use with various methods for retrieving tissue specimens during a surgical procedure and, in particular, a minimally invasive surgical procedure including, for example, cholecystectomies, appendectomies, nephrectomies, colectomies, splenectomies, laparoscopic hysterectomies, and the like.

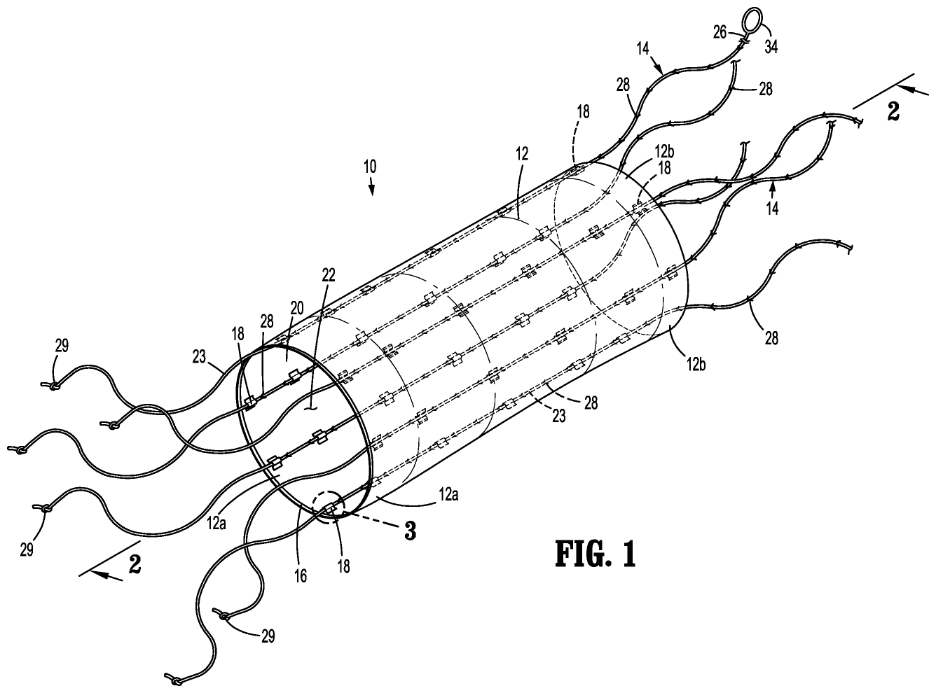

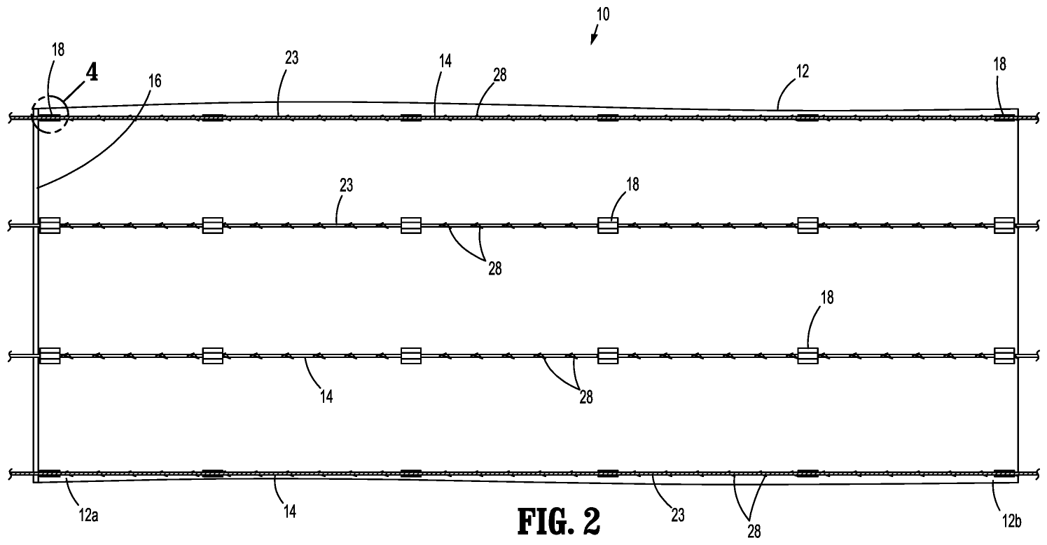

FIGS. 1-4 illustrate a specimen retrieval device 10 according to aspects of the disclosure. The specimen retrieval device 10 includes a sleeve 12, barbed sutures 14, a band 16, and guide members 18. The sleeve 12 has a tubular configuration with open distal and proximal ends 12a and 12b and includes an inner surface 20 that defines a cavity 22. In aspects of the disclosure, the sleeve 12 is formed of a collapsible biocompatible material such as polyurethane, latex, or ripstop nylon. Alternately, other materials of construction can be used to form the sleeve 12.

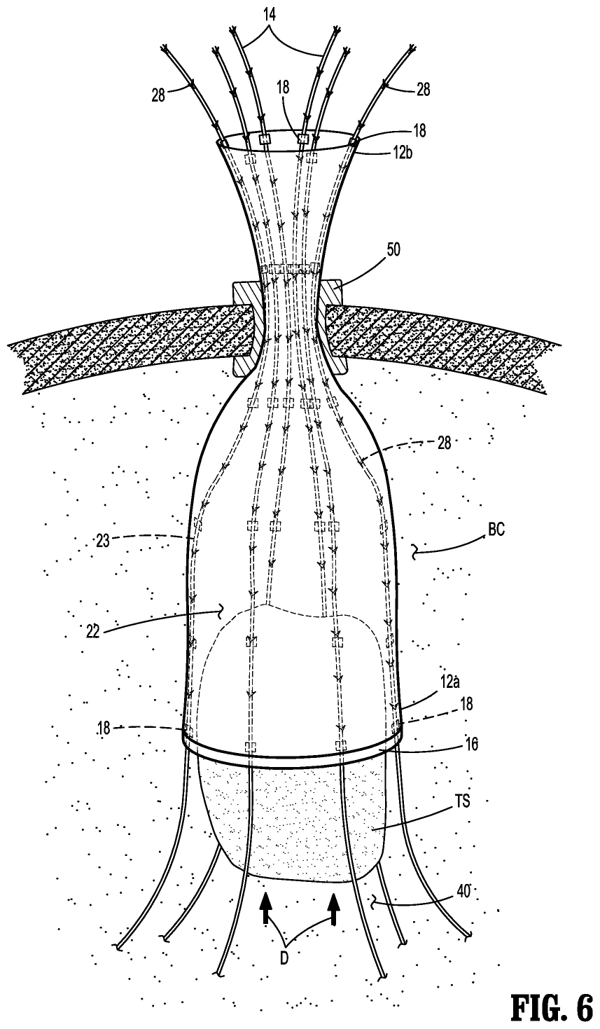

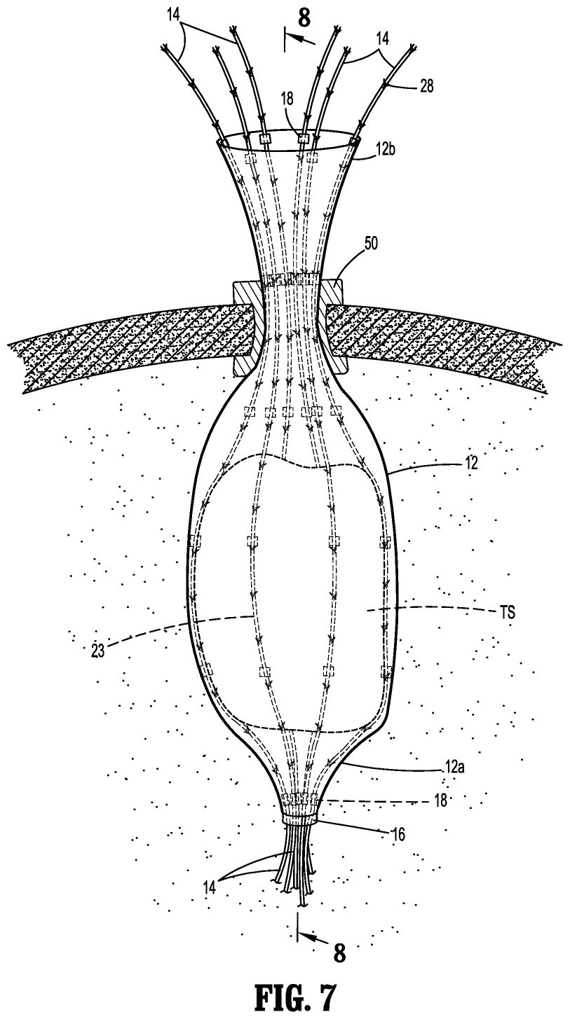

The band 16 is secured about the open distal end 12a of the sleeve 12 and can transition or be transitioned from an open configuration (FIG. 6) to a contracted configuration (FIG. 7). In the open configuration, the band 16 defines a first diameter and is secured to the distal portion of the sleeve 12 to retain the distal end 12a of the sleeve 12 in an open configuration. In the open configuration, a tissue specimen “TS” can pass through the open distal end 12a of the sleeve 12 into the cavity 22 of the sleeve 12. In the contracted configuration, the band 16 defines a second diameter that is smaller than the first diameter to close or partially close the open distal end 12a of the sleeve 12 and contain the tissue specimen “TS” within the cavity 22 of the sleeve 12.

The band 16 has a circular shape and can be secured to the distal portion of the sleeve 12 using a variety of different fastening techniques. For example, the band 16 can be secured within a cuff (not shown) formed in the distal end 12a of the sleeve 12 or welded to the distal end 12a of the sleeve 12. In aspects of the disclosure, the band 16 is formed from a resilient or flexible shape memory material such as Nitinol that can transition from the open configuration to the contracted configuration in response to a change in ambient conditions such as temperature or pressure. In some aspects of the disclosure, the band 16 is formed from a material that transitions from the open configuration to the contracted configuration in response to a change in temperature that occurs upon exposure of the band 16 to body temperatures. Alternately, other materials can be used to form the band 16. These materials can be selected to automatically transition from the open configuration to the contracted configuration in response to a change in ambient conditions, or in response to an external stimulus or input such as an electrical charge.

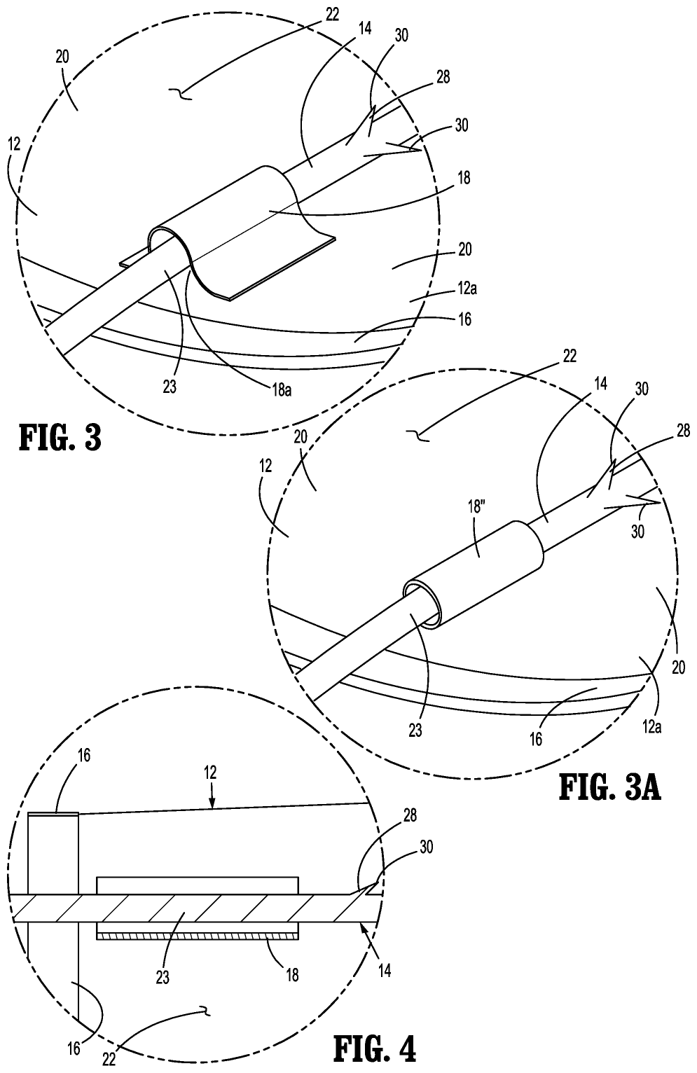

FIG. 3 illustrates one of the guide members 18 of the specimen retrieval device 10 which includes a flap of material that is secured at both ends to the inner surface 20 of the sleeve 12 to define a channel 18a between the inner surface of the sleeve 12 and the flap 18. The channel 18a of each of the guide members 18 receives one of the barbed sutures 14 such that the barbed sutures 14 can move or slide within the channels 18a of the guide members 18. In aspects of the disclosure, two or more of the guide members 18 are longitudinally aligned along the inner surface 20 of the sleeve 12 to receive each of the barbed sutures 14.

In some aspects of the disclosure, some of the guide members 18 are secured on a distal portion of the sleeve 12 and some of the guide members 18 are secured on a proximal portion of the sleeve 12, and each of the guide members 18 on the distal portion of the sleeve 12 is longitudinally aligned with one of the guide members 18 on the proximal portion of the sleeve 12. This configuration of the guide members 18 supports the barbed sutures 14 such that the barbed sutures extend longitudinally along the inner surface 22 through the cavity 22 of the sleeve 12.

FIG. 3A illustrates an alternative version of the guide member 18 shown as guide member 18″. The guide members 18″ have a tubular configuration (FIG. 3) and are secured to the inner surface 20 of the sleeve 12. Each of the guide members 18″ receives one of the barbed sutures 14 as described in further detail below to allow the barbed sutures 14 to move or slide within the guide members 18″.

Each of the barbed sutures 14 has an elongate body 23, a distal portion 24, a proximal portion 26 (FIG. 1), and barbs 28 positioned along the elongate body 23. The distal portions 24 of the barbed sutures 14 extend through channels 18a of the guide members 18 on the distal portion of the sleeve 12 and extend distally of the sleeve 12. The proximal portions 26 of the barbed sutures 14 extend through the channels 18a of the guide members 18 on the proximal portion of the sleeve 12 and extend proximally of the sleeve 12. The barbed sutures 14 form a cage-like or whisk configuration within the cavity 22 of the sleeve 12.

In aspects of the disclosure, the barbed sutures 14 of the specimen retrieval device 10 number from about 4 to about 12 barbed sutures 14, in some aspects of the disclosure from about 6 to about 10 barbed sutures 14, and in other aspects about 6 barbed sutures 14. Alternately, other numbers of barbed sutures 14 are envisioned. In aspects of the disclosure, the elongate body 23 of the barbed sutures 14 are dimensioned to slide within the respective guide members 18 but the barbs 28 of the barbed sutures have a width to prevent passage of the barbs 28 through the guide members 18.

The barbs 28 on the barbed sutures 14 extend from the elongate body 23 and include tips 30 that extend from the elongate body 23 in a proximal direction. The orientation of the tips 30 of the barbs 28 is such that when the proximal portions 26 of the barbed threads 20 are pulled proximally by a clinician, the barbs 28 engage and grab a tissue specimen “TS” to withdraw the tissue specimen “TS” or a portion of the tissue specimen from a body cavity “BC” of a patient.

The barbed sutures 14 may be formed of any suitable biocompatible material. For example, suitable biocompatible materials include polymeric materials such as those derived from lactones including lactide, glycolide, caprolactone, and valerolactone; carbonates such as trimethylene carbonate, tetramethylene carbonate, and the like; dioxanones such as 1,4-dioxanone; 1, dioxepanones such as 1,4-dioxepan-2-one and 1,5-dioxepan-2-one; ethylene glycol; ethylene oxide; ester amides; γ-hydroxyvalerate; β-hydroxypropionate; alpha-hydroxy acid; hydroxybutyrates; poly (ortho esters); hydroxy alkanoates; tyrosine carbonates; polyimide carbonates; polyimino carbonates such as poly (bisphenol A-iminocarbonate) and poly (hydroquinone-iminocarbonate); polyurethanes; polyanhydrides; and copolymers and combinations thereof.

The barbs 28 may be formed on the barbed sutures 14 by any method within the purview of those skilled in the art, including cutting or similar processes utilized for forming barbs on sutures or similar thread-like devices. In aspects, suitable barbed sutures utilized with the specimen retrieval device 10 include those sold commercially as V-LOC™ sutures from Medtronic (North Haven, CT).

The proximal portion 26 of each of the barbed sutures 14 includes a gripping member, e.g., a loop 34, that can be gripped by a clinician to withdraw one or more of the barbed sutures 14 and a portion of the tissue specimen “TS” from the body cavity of the patient. The gripping member 34 can have a variety of different grip configurations. In aspects of the disclosure, the distal portion of each of the barbed sutures 14 that is positioned distally of the guide member 18 on the distal portion of the sleeve 12 does not include barbs 28. In some aspects of the disclosure, the distal portion of each of the barbed sutures 14 includes knots 29 (FIG. 1) to prevent passage of a distal end of each of the barbed sutures 14 through a channel 18a of a respective guide member 18. When the knot 29 of a barbed suture 14 engages the guide member 18 as the barbed suture 14, is pulled proximally, the sleeve is also pulled proximally.

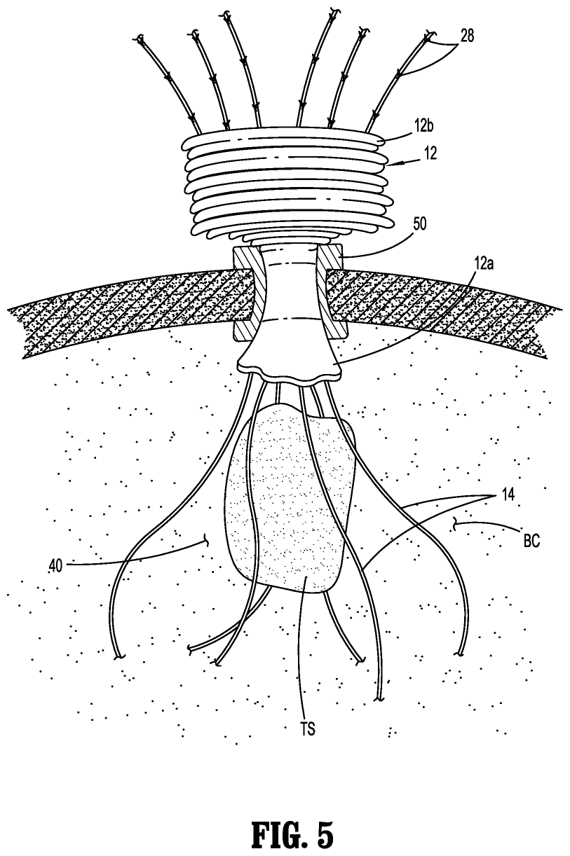

In use, as depicted in FIGS. 5-10, the specimen retrieval device 10 including the band 16, the distal portion of the sleeve 12, and the distal portions of the barbed sutures 14 are introduced into a patient's body cavity “BC” through an access port 50. In aspects of the disclosure, the specimen retrieval device 10 can be introduced through the access port 50 in a rolled or folded configuration and subsequently unrolled or unfolded using, e.g., a grasper that is introduced into the body cavity “BC” through a separate access port (not shown). The barbed sutures 20 are in a relaxed configuration, permitting placement of a tissue specimen “TS” in the direction of arrows “D” in FIG. 6 within a space 40 (FIG. 5) defined within the confines of the barbed sutures 14 in a position aligned with the open distal end 12a of the sleeve 12 and an opening of the band 16. The tissue specimen “TS” can now be positioned through the band 16 and the open distal end 12a of the sleeve 12 into the cavity 22 defined by the sleeve 12 (FIG. 6). This may also be accomplished with the assistance of a grasper (not shown).

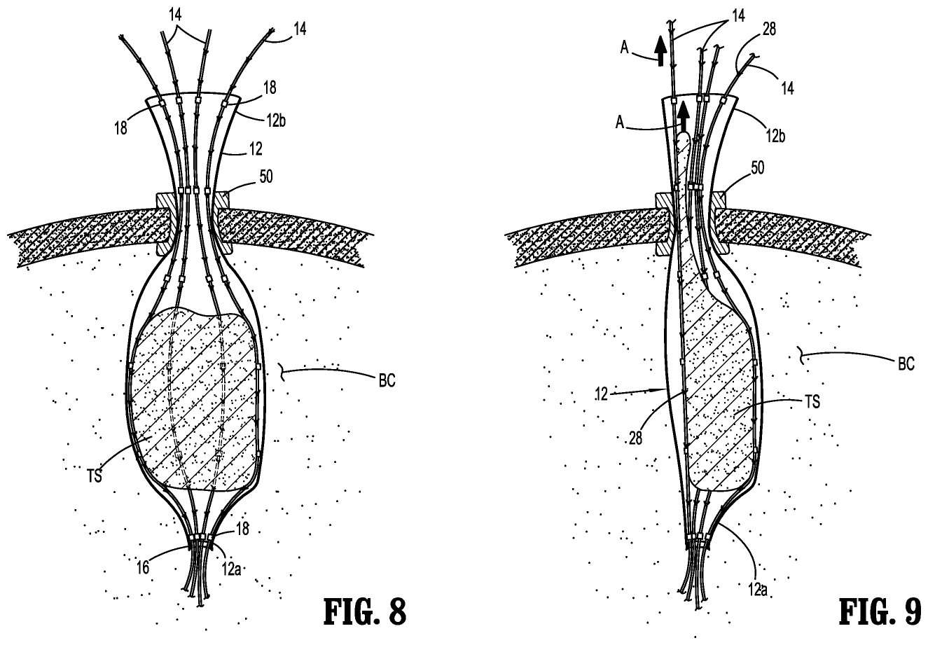

As illustrated in FIGS. 7 and 8, once the tissue specimen “TS” is positioned within the cavity 22 defined by the sleeve 12 and within the confines 40 of the barbed sutures 14, the band 16 transitions or is transitioned from the open configuration to the contracted configuration to confine the tissue specimen “TS” within the sleeve 12 (FIG. 7). Next, the clinician operates the specimen retrieval device 10 by pulling the proximal portions 26 (FIG. 1) of one or more of the barbed sutures 14 in a proximal direction as illustrated by arrow “A” in FIG. 9. When the barbed sutures 14 are pulled proximally and tensioned, the barbed sutures 14 move inwardly to move the barbs 28 into engagement with the tissue specimen “TS” to grip the tissue specimen “TS”. After the barbs 28 of the barbed suture or sutures 14 engage the tissue specimen “TS”, further proximal movement of the one or more barbed sutures 14 withdraws a portion of the tissue specimen “S” and a portion of the sleeve 12 through the access port 50.

In some aspects of the disclosure, the barbs 28 of the barbed sutures 14 have a width that is larger than a size of the channel 18a (FIG. 3) of the guide members 18. As such, proximal movement of the barbed sutures 14 causes proximal movement of the sleeve 12 when the barbs 28 engage the guide members 18.

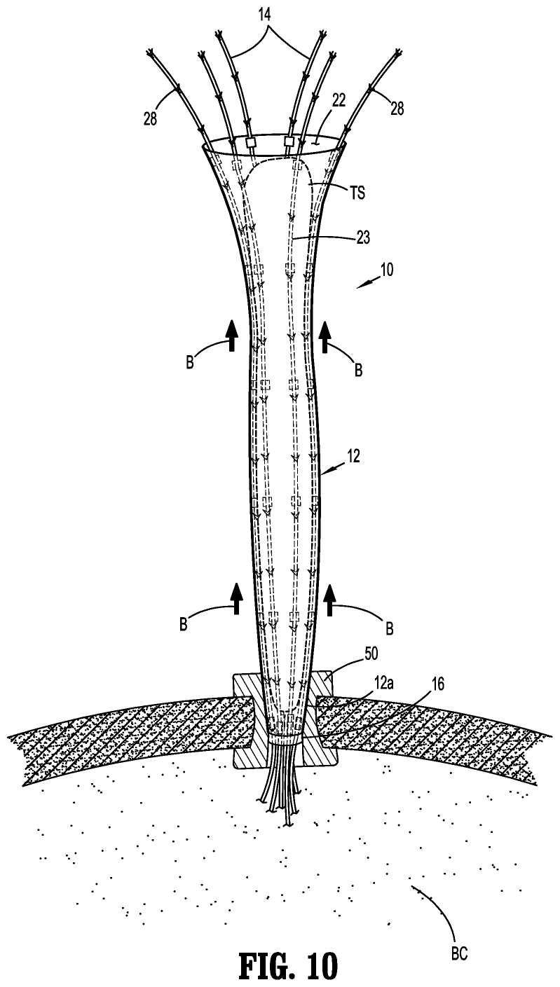

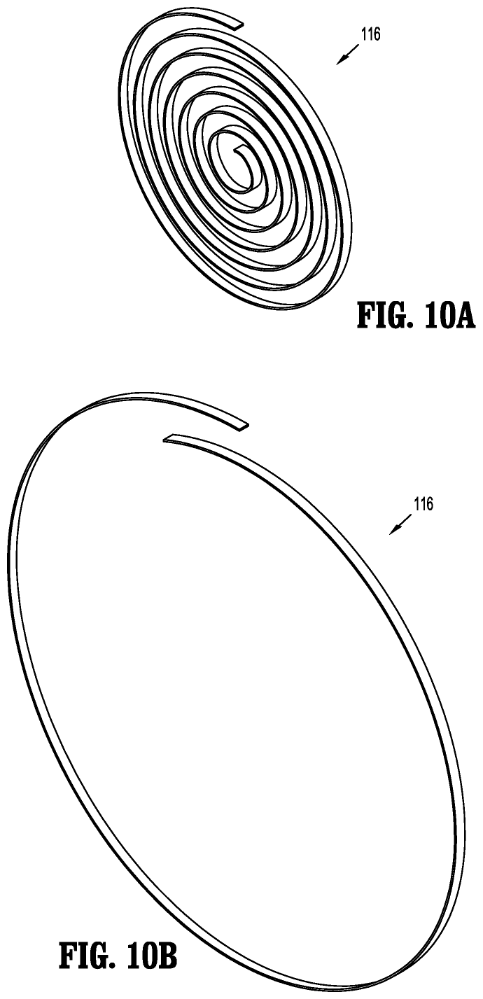

This process can be sequentially repeated to remove the entire tissue specimen “TS” from the body cavity “BC” through the access port 50 by pulling on the proximal portion 26 (FIG. 1) of one or more of the barbed sutures 14 in the direction of arrows “B” in FIG. 10. Alternately, the proximal portions 26 of all the barbed sutures 14 can be pulled simultaneously to remove the sleeve 12 and the tissue specimen “TS” from the body cavity “BC” through the access port 50.

In aspects of the disclosure, the proximal portions 26 (FIG. 1) of the barbed sutures 14 may be pulled by a clinician in a clockwise or counterclockwise pattern, or a cross pattern, to pull portions of the tissue specimen “TS” within the sleeve 12 through the access port 50 from the body cavity “BC”. In some aspects of the disclosure, diametrically opposed barbed sutures 14 may be simultaneously pulled proximally in the direction of arrows “B” in FIG. 10 to compress the tissue specimen “TS” between the opposed barbed sutures 14. Alternatively, the clinician may pull the proximal portions of the barbed sutures 14 of the specimen retrieval device 10 on only one side of the specimen retrieval device 10 to assist in advancing dense anatomy of a tissue specimen “TS” and removal of the specimen retrieval device 10 from the patient's body cavity “BC”.

As shown in FIG. 10, once the bulk of the tissue specimen “TS” has been pulled through the access port 50, the proximal portion of the sleeve 12 and the proximal portions of the barbed threads 14 are externalized by pulling the remainder of the barbed sutures 14 in the direction of arrows “B” through the access port 50. As the barbed sutures 14 are pulled from the body cavity “BC”, the sleeve 12 is also pulled from the body cavity “BC”.

Once the specimen retrieval device 10 has been removed from the patient's body, the tissue specimen “TS” may be removed from the sleeve 12 for further examination.

Benefits of the disclosed specimen retrieval device include their use in the removal of large tissue specimens from a body cavity. Current extraction techniques rely on pulling on a specimen or dragging an unsupported bag through an incision site. The pulling technique requires continuous grip strength when forceps are used and has the risk of tearing the specimen during extraction. The bag technique results in a large build up of tissue mass and fluid at the end of the bag, which increases in pressure until the entire bag is pulled through (or a burst occurs). The specimen retrieval device of the disclosure allows for removal of larger tissue specimens through smaller incisions. Specimen extraction through smaller incisions provides the benefit of reduced trauma and less scarring.

In aspects of the disclosure, the band 16 may have any shape or configuration that can transition between open and contracted configurations. In one alternate version of the band 16 illustrated in FIGS. 10A and 10B, the band 116 has a helical or spiral configuration in the contracted configuration of the band 116 and a substantially circular configuration in the open configuration of the band 116. In some aspects of the disclosure, the band 116 is received in a cuff of the 12 (FIG. 1) and in the contracted configuration, winds up inside the cuff. In certain aspects of the disclosure, the band 116 is formed of a super elastic material, e.g., nitinol, that can be rolled up to pass through an incision, and transition to the open configuration upon exposure to body temperature.

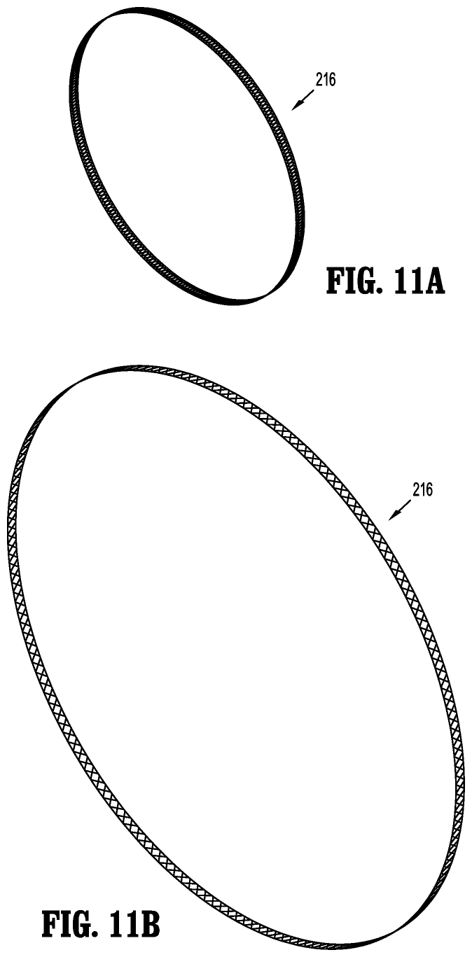

FIGS. 11A and 11B illustrate another alternate version of the band shown generally as band 216. The band 216 has a lattice structure that transitions between contracted and open configurations. The diameter of an opening defined by the band 216 increases as the band 216 transitions from the contracted to the open configuration. In some aspects of the disclosure, the band 216 is formed of a super elastic material, e.g. nitinol, that is normally in the contracted configuration but transitions to the open configuration upon exposure to body temperature.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto. Additionally, it is envisioned that the elements and features illustrated or described in connection with one exemplary aspect may be combined with the elements and features of another disclosed aspect without departing from the scope of the disclosure, and that such modifications and variations are also intended to be included within the scope of the disclosure. Accordingly, the disclosure is not to be limited by what has been particularly shown and described, except as indicated by the appended claims.

What is claimed is:

1. A specimen retrieval device comprising:
a sleeve defining a cavity and having an first open end, an second open end, and an inner surface defining the cavity;
a band supported on the sleeve about the first open end of the sleeve, the band being transitionable from an open configuration to a contracted configuration; and
barbed sutures extending through the sleeve, each of the barbed sutures having an elongate body, a distal portion, a proximal portion, and barbs that are spaced along at least a portion of the elongate body, wherein each of the barbed sutures is coupled to the inner surface of the sleeve and extends from the first open end of the sleeve to the second open end of the sleeve.

2. The specimen retrieval device of claim 1, further including guide members secured to the inner surface of the sleeve, each of the guide members defining a channel that receives one of the barbed sutures to couple the barbed suture to the inner surface of the sleeve.

3. The specimen retrieval device of claim 2, wherein the sleeve includes a distal portion and a proximal portion, and the guide members are supported on the distal and proximal portions of the sleeve.

4. The specimen retrieval device of claim 3, wherein each of the guide members on the distal portion of the sleeve is longitudinally aligned with one of the guide members on the proximal portion of the sleeve.

5. The specimen retrieval device of claim 4, wherein the guide members that are longitudinally aligned on the distal and proximal portions of the sleeve receive one of the barbed sutures such that the one of the barbed sutures extends longitudinally along the inner surface of the sleeve.

6. The specimen retrieval device of claim 1, wherein the band is formed from a shape memory material that transitions from the open configuration to the contracted configuration in response to a change in ambient conditions.

7. The specimen retrieval device of claim 6, wherein the shape memory material is selected to transition from the open configuration to the contracted configuration in response to a change temperature.

8. The specimen retrieval device of claim 1, wherein the barbed sutures include from about 4 to about 12 barbed sutures.

9. The specimen retrieval device of claim 8, wherein the barbed sutures include from about 6 to about 10 barbed sutures.

10. The specimen retrieval device of claim 1, wherein the plurality of barbed threads are formed of a biocompatible material selected from polymeric materials derived from lactones, carbonates, dioxanones, ethylene glycol, ethylene oxide, ester amides, γ-hydroxyvalerate, β-hydroxypropionate, alpha-hydroxy acid, hydroxybutyrates, poly (ortho esters), hydroxy alkanoates, polyurethanes, polyanhydrides, copolymers thereof, or combinations thereof.

11. The specimen retrieval device of claim 7, wherein the shape memory material is nitinol.

12. The specimen retrieval device of claim 1, wherein the barbed sutures are spaced about the inner surface of the sleeve.

13. The specimen retrieval device of claim 1, wherein the sleeve is formed of a biocompatible material selected from nylon, polyurethane, and latex.

14. The specimen retrieval device of claim 2, wherein the barbs of the barbed suture have a width that is greater than a size of the channels of the guide members.

15. The specimen retrieval device of claim 1, wherein the distal portion of each of the barbed sutures extends from the first open end of the sleeve and the proximal portion of each of the barbed sutures extends from the second open end of the sleeve.

16. The specimen retrieval device of claim 1, wherein each of the barbs extends from the elongate body of the barbed suture in a proximal direction.

17. The specimen retrieval device of claim 1, wherein the band has a spiral configuration when the band is in the contracted configuration.

18. The specimen retrieval device of claim 1, wherein the band has a lattice structure.

19. A method of removing tissue from a patient comprising:
introducing a specimen retrieval device having a sleeve having open distal and proximal ends and barbed sutures through an access port into a body cavity of a patient in a folded or rolled configuration;
unfolding or unrolling the sleeve and barbed sutures of the specimen retrieval within the body cavity of the patient;
positioning a tissue specimen into a cavity defined by the sleeve through a distal open end of the sleeve within confines of the barbed sutures;
moving the distal open end of the sleeve from an open configuration to a contracted configuration; and
pulling a proximal portion of at least one of the barbed sutures so that barbs of the barbed sutures engage the tissue specimen and pull at least a portion of the tissue specimen through the access port.

20. The method of claim 19, wherein pulling the proximal portion of at least one of the barbed sutures is repeated until each of the barbed sutures is pulled proximally to remove the sleeve and the tissue specimen through the access port from the body cavity.
